(19) United States
(12) Patent Application Publication
(10) Pub. No.: US 2025/0285440 A1
(43) Pub. Date: Sep. 11, 2025

(54) DATA COLLECTION APPARATUS, METHOD, AND STORAGE MEDIUM

(71) Applicant: KABUSHIKI KAISHA TOSHIBA, Tokyo (JP)

(72) Inventors: Yukinobu Sakata, Kawasaki Kanagawa (JP); Kenta Umene, Fuchu Tokyo (JP); Saori Asaka, Yokohama Kanagawa (JP); Ryusuke Hirai, Tokyo (JP)

(73) Assignee: KABUSHIKI KAISHA TOSHIBA, Tokyo (JP)

(21) Appl. No.: 19/075,438

(22) Filed: Mar. 10, 2025

(30) Foreign Application Priority Data
Mar. 11, 2024 (JP) 2024-037535

(51) Int. Cl. G06V 20/40; G06T 7/50; G06T 7/90

(52) U.S. Cl. CPC G06V 20/44; G06T 7/50; G06T 7/90; G06V 2201/07

(57) ABSTRACT

A data collection apparatus includes a processing circuitry that is configured to: detect a landmark from an input image that has been input; set, based on a relative position with respect to the landmark, either a log collection range or an event detection position; detect an event at the event detection position; and collect data of the log collection range in a case where the event is detected.

### CROSS-REFERENCE TO RELATED APPLICATION

This application is based upon and claims the benefit of priority from Japanese Patent Application No. 2024-037535, filed Mar. 11, 2024, the entire contents of which are incorporated herein by reference.

### FIELD

Embodiments described herein relate generally to a data collection apparatus, a method, and a storage medium.

### BACKGROUND

In the heavy particle therapy using the heavy particle therapy apparatus, it is necessary to accurately aim the heavy particle beam with which the affected area is irradiated in advance in order not to damage the normal tissue. Thus, a user makes adjustments prior to treatment such that the beam is transported to the center of the treatment room. The position of the beam is controlled by electromagnets disposed at a plurality of positions along a path from an accelerator to the treatment room. The beam accelerated by the accelerator is transported to the treatment room through a beam delivery tube. The user checks the position of the beam passing through the positions where the electromagnets are disposed and adjusts parameters of each of the electromagnets such that the beam passes through the center of the delivery tube.

In general, since the positions at which the electromagnets are to be disposed is equal to or more than 10, the user visually adjusts several tens of parameters. In addition, adjustment is required for each intensity and irradiation angle of the beam. For example, in a case where the beam intensity can be adjusted in 600 steps and the beam angle can be adjusted in 360 steps, adjustments of 600×360=216000 times are required.

In order to reduce a burden on the user, a technique for automating beam adjustment is desired. In order to automate the beam adjustment, it is necessary to collect a work log when the user actually performs the beam adjustment. However, in general, applications used for adjusting parameters of an electromagnet and confirming a beam position are different, and a function of collecting a work log is not installed in advance in these applications in many cases. Therefore, it is difficult to effectively collect the work log of the user.

In addition, there is a data collection apparatus that collects actual work data executed by a work terminal in order to automate beam adjustment. Such a data collection apparatus can collect actual work data without modifying the work terminal by, for example, acquiring an image displayed on a display of the work terminal and analyzing the acquired image to collect the work data.

In such a data collection apparatus, there is a case where data cannot be properly collected if the user's operation intervenes during data collection. For example, in a case where the position or layout of the window changes during the work, accuracy of image analysis is deteriorated. In addition, since the user needs to determine the data collection timing by himself/herself, the collection accuracy changes depending on the user's operation.

### BRIEF DESCRIPTION OF DRAWINGS

FIG. 1 is a diagram illustrating an example of a configuration of a data collection apparatus according to a first embodiment.

FIG. 2 is a flowchart showing a processing procedure of data collection processing according to the first embodiment.

FIG. 3 is a diagram illustrating a flow of data in the data collection processing according to the first embodiment.

FIG. 4 is a diagram illustrating an example of an input image according to the first embodiment.

FIG. 5 is a diagram in which a position of the display window in FIG. 4 has changed.

FIG. 6 is a diagram illustrating an example of an exception image according to the first embodiment.

FIG. 7 is a diagram illustrating an example of collected data according to the first embodiment.

FIG. 8 is a diagram illustrating an example of the input image according to a first modification of the first embodiment.

FIG. 9 is a diagram illustrating an example of the input image according to a second modification of the first embodiment.

FIG. 10 is a diagram illustrating an example of a configuration of a data collection apparatus according to a second embodiment.

FIG. 11 is a flowchart showing a processing procedure of data collection processing according to the second embodiment.

FIG. 12 is a diagram illustrating a flow of data in the data collection processing according to the second embodiment.

### DETAILED DESCRIPTION

In general, according to one embodiment, a data collection apparatus includes a processing circuitry that is configured to: detect a landmark from an input image that has been input; set, based on a relative position with respect to the landmark, either a log collection range or an event detection position; detect an event at the event detection position; and collect data of the log collection range in a case where the event is detected.

Hereinafter, embodiments of a data collection apparatus, a method, and a program will be described in detail with reference to the drawings. In the following description, components having substantially the same functions and configurations are denoted by the same reference numerals, and redundant description will be made only when necessary.

### First Embodiment

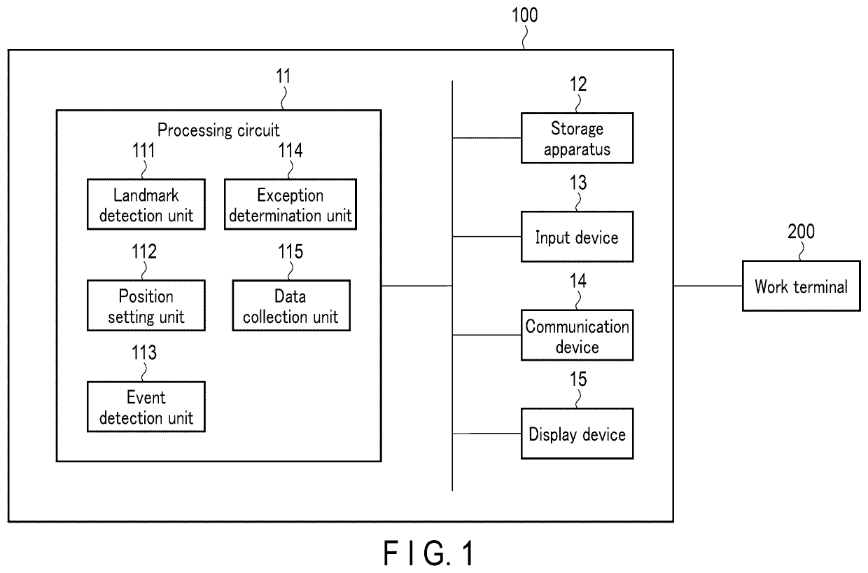

FIG. 1 is a diagram illustrating an example of a configuration of a data collection apparatus 100 according to a first embodiment. The data collection apparatus 100 is connected to a work terminal 200. The connection method between the data collection apparatus 100 and the work terminal 200 may be a wired connection, a wireless connection, or a connection via a network.

The work terminal 200 is a terminal apparatus for a worker to perform a predetermined work. The work terminal 200 includes an input interface such as a keyboard and a mouse, a console apparatus including a processing circuitry, a display, and the like. The work terminal 200 is, for example, a general PC terminal or a tablet terminal. The worker performs a work using, for example, a prescribed application installed in the work terminal 200.

The work terminal 200 is, for example, an apparatus for adjusting a heavy particle beam in a heavy particle therapy apparatus. In this case, for example, the worker starts a parameter input application and a beam observation application displayed on the display of the work terminal 200, and adjusts parameters using the parameter input application so that a beam distribution displayed in the beam observation application has a desired distribution.

The data collection apparatus 100 collects work data in the work terminal 200. The data collection apparatus 100 collects a work log of the work terminal 200 by continuously acquiring a display image on the work terminal 200. The data collection apparatus 100 is, for example, a general notebook PC terminal or a tablet terminal. The work log is information on a work executed using the work terminal 200. The work log includes, for example, an operation log, a parameter change history, an analysis result of a display image, and the like. The work log may be referred to as work data or collected data. The data collection apparatus 100 may also be referred to as a data collection unit.

The data collection apparatus 100 is a computer including processing circuitry 11, a storage apparatus 12, an input device 13, a communication device 14, and a display device 15. Data communication among the processing circuitry 11, the storage apparatus 12, the input device 13, the communication device 14, and the display device 15 is performed via a bus. The input device 13 and the display device 15 may not be provided.

The processing circuitry 11 includes a processor such as a central processing unit (CPU) and a memory such as a random access memory (RAM). The processing circuitry 11 includes a landmark detection unit 111, a position setting unit 112, an event detection unit 113, an exception determination unit 114, and a data collection unit 115. By executing the program, the processing circuitry 11 realizes a landmark detection function, a position setting function, an event detection function, an exception determination function, and a data collection function by the above-described units.

The storage apparatus 12 includes a read only memory (ROM), a hard disk drive (HDD), a solid state drive (SSD), an integrated circuit memory apparatus, and the like. The storage apparatus 12 stores a program and the like.

The program is stored in a non-transitory computer-readable recording medium such as the storage apparatus 12. The program may be implemented as a single program that describes all the functions of the above units, or may be implemented as a plurality of modules divided into several functional units. Each of the above units may be implemented by an integrated circuit such as an Application Specific Integrated Circuit (ASIC). In this case, the above units may be mounted on a single integrated circuit, or may be individually mounted on a plurality of integrated circuits.

The input device 13 inputs various commands from an operator. As the input device 13, a keyboard, a mouse, various switches, a touch pad, a touch panel display, and the like can be used. An output signal from the input device 13 is supplied to the processing circuitry 11.

The communication device 14 is an interface for performing data communication with an external device connected to the data collection apparatus 100 via a network. For example, the communication device 14 performs data communication with the work terminal 200.

The display device 15 displays various types of information. As the display device 15, a CRT (Cathode-Ray Tube) display, a liquid crystal display, an organic EL (Electro Luminescence) display, an LED (Light-Emitting Diode) display, a plasma display, or any other display known in the art can be appropriately used. Furthermore, the display device 15 may be a projector.

Next, each function executed by each unit of the processing circuitry 11 will be described in detail.

The landmark detection unit 111 detects a landmark from the input image that has been input to the work terminal 200. At this time, the landmark detection unit 111 first continuously acquires image data of a display image displayed on the display of the work terminal 200. At this time, a display screen displayed on the display of the work terminal 200 may be acquired, or an output signal that has been output from the console apparatus of the work terminal 200 to the display in order to display the display screen on the display may be acquired. Hereinafter, the image data acquired from the work terminal 200 is referred to as an input image.

Thereafter, the landmark detection unit 111 detects, from the input image, a position of a landmark designated in advance. The landmark is used to determine a log collection range and an event detection position. It is desirable that a positional relationship of the landmark with the log collection range or the event detection position does not change even if the layout of the input image or the position of the application on the input screen changes, and that the landmark is a unique pattern in the input image. The unique pattern is a portion having a specific color or a specific shape in the input image. For example, an icon or tab having a specific color or a specific shape can be used as the landmark.

The log collection range is an area where data is collected in the input image, and is an area used for collecting the work log. The log collection range is, for example, a range in which an image is captured. The log collection range may be referred to as a data collection range or a capture range. The log collection range is, for example, a range including a parameter display area in a parameter input application or a range including a display area of an energy distribution in a beam observation application.

The event detection position is a position where an event for determining a timing to execute data collection is detected. As the event detection position, for example, a specific operation button or a display portion that displays a specific parameter can be used. For example, it is possible to detect an occurrence of an event in which a condition changes based on a change in which an icon of the specific operation button changes to an operated state. It is also possible to detect an occurrence of an event for adjusting a parameter based on a change in a value of the parameter on the display portion that displays the specific parameter.

For the detection of the landmark, a method using template matching or a filter response, for example, can be used. The method using the template matching is a method in which an image of a preset landmark is held as a template, and a portion matching the held image is detected from the screen. The method using the filter response is a method in which, in a case where the shape of the landmark is known, a filter that reacts to the shape of the landmark is designed in advance, and the landmark is detected from the screen by applying the filter to the image. Note that it is possible to detect a plurality of landmarks. For example, a landmark for setting the log collection range and a landmark for setting the event detection position may be separately detected.

The position setting unit 112 sets the log collection range and the event detection position based on the position of the landmark detected by the landmark detection unit 111. At this time, the position setting unit 112 sets the log collection range based on a relative position of the log collection range with respect to the landmark, and sets the event detection position based on a relative position of the event detection position with respect to the landmark. A positional relationship between the landmark and the log collection range and a positional relationship between the landmark and the event detection position are preset by a user and stored in the storage apparatus 12.

The event detection unit 113 detects an event at the event detection position. The event is used to determine a timing for collecting data. The event is, for example, a specific operation or a screen transition by the specific operation. Examples of the method of detecting an event include a method of detecting a change in an image and a method of detecting a specific change pattern in an image.

The method for detecting a change in an image can be used in a case where time-series information is collected by continuously acquiring input images. For example, it is possible to determine that an event has occurred in a case where, by constantly storing an image pattern of the event detection position and calculating a difference between the previous image pattern and the current image pattern, the difference exceeds a threshold value. In addition, in a case where a number or a character is included in the image pattern at the event detection position, the number or the character may be analyzed and stored, and a change in the number or the character may be detected as an event.

The method of detecting a specific image pattern can be used in a case of detecting an operation of displaying a specific image pattern. For example, the image pattern at the event detection position is compared with the specific image pattern stored in advance, and it is determined that an event has occurred in a case where the image patterns match. For example, in a case where a specific button is set as the event detection position, and by selecting the button, the image pattern of the selected button changes to a specific image pattern, it is possible to detect that the user has pressed the specific button on the display screen by detecting the occurrence of the image pattern of the selected button as the event.

The exception determination unit 114 analyzes an image of the log collection range at the timing at which the event is detected, and determines whether or not the image of the log collection range is an exception image. The exception image is data having a high possibility that in a case where the image of the log collection range is analyzed, an analysis result takes an exception value or an outlier. For example, in a case in which another display portion is displayed over the log collection range and a data display area of a collection target is hidden by the another display portion, it is determined that the image in the log collection range is the exception image. The another display portion is, for example, a window of another application, a file transfer dialog, a pull-down, a parameter setting GUI, a mouse cursor, or the like. Alternatively, a character and a number in the image of the log collection range may be detected using image analysis, and the image of the log collection range may be determined to be the exception image in a case where no character or number are displayed or in a case where an anomalous character or number is displayed. In addition, a reference image serving as a reference may be stored in advance, a degree of similarity between the image in the log collection range and the reference image may be calculated, and in a case where the degree of similarity is low, it may be determined that the image is the exception image.

The data collection unit 115 collects data in the log collection range at the timing at which the event is detected. At this time, the data collection unit 115 excludes an image determined to be the exception image by the exception determination unit 114, and only collects the image not determined to be the exception image. The collected image is stored in the storage apparatus 12, for example.

The data collection unit 115 may collect a result of the image analysis on the collected image. For example, characters and numbers obtained by the image analysis may be collected, or the position, distribution, shape, and the like of the object in the image may be analyzed and collected.

### (Data Collection Processing)

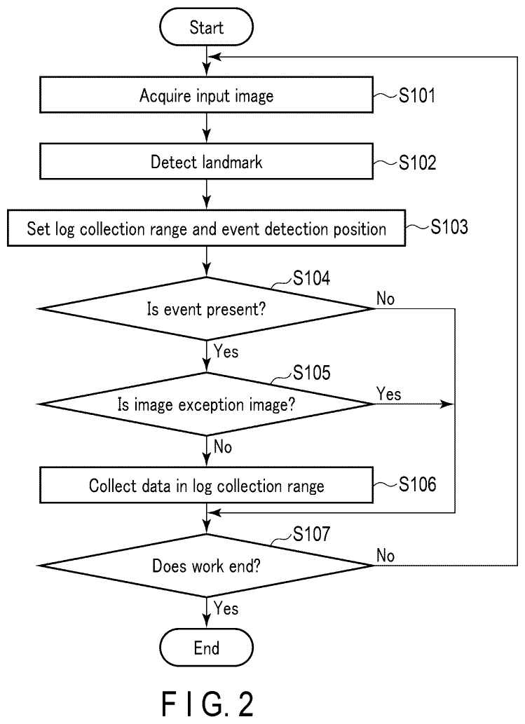

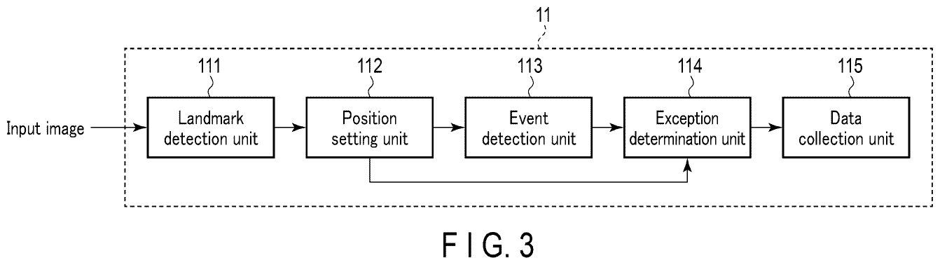

Next, an operation of data collection processing executed by the data collection apparatus 100 will be described. FIG. 2 is a flowchart showing an example of a procedure of the data collection processing. FIG. 3 is a diagram illustrating an example of a flow of data in the data collection processing. Note that the processing procedure in each processing described below is merely an example, and each processing can be appropriately changed as much as possible. In addition, regarding the processing procedure described below, it is possible to appropriately omit, replace, and add a step according to the embodiment.

Here, a case in which a work log of a work using a beam confirmation application is collected will be described. The work terminal 200 is a terminal that observes a heavy particle beam passing near each electromagnet of a heavy particle therapy apparatus using a beam observation application, and a case in which an energy distribution of the heavy particle beam is collected using the data collection apparatus 100 will be described as an example. The data collection processing is executed in a state where the data collection apparatus 100 is connected to the work terminal 200.

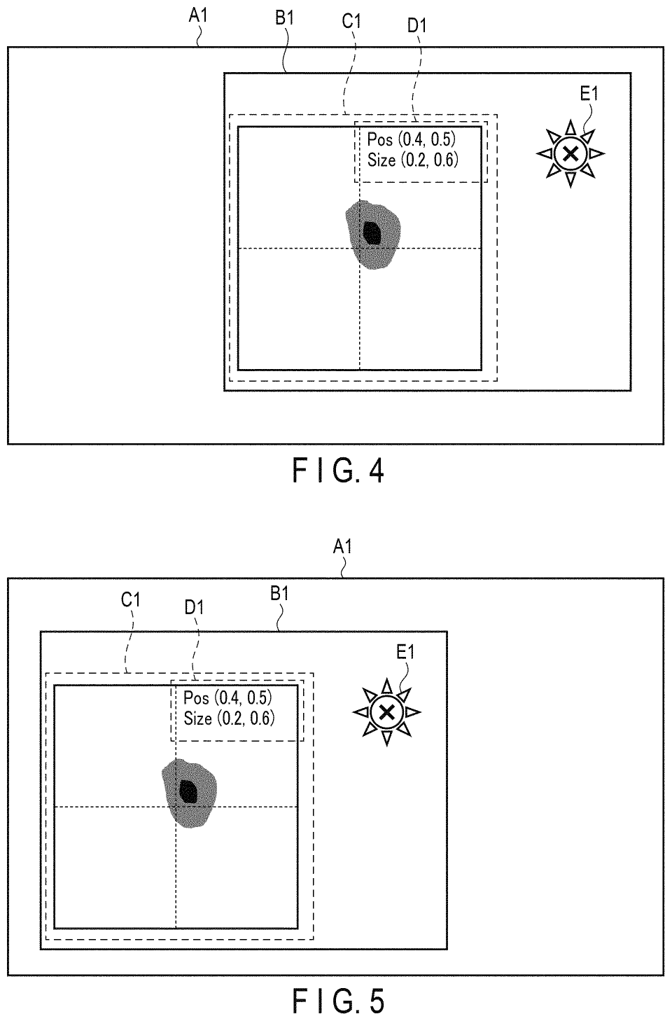

FIGS. 4 and 5 are diagrams illustrating examples of an input image A1 acquired from the work terminal 200. The input image A1 is a display image output to the display of the work terminal 200. In the examples of FIGS. 4 and 5, a display window B1 of the beam observation application is displayed in the input image A1. FIG. 5 illustrates a state in which a position of the display window B1 has changed from the state illustrated in FIG. 4.

Here, a display area C1 of an energy distribution of the beam (hereinafter, referred to as an energy distribution display portion) is set as the log collection range, and a display area D1 of an analysis result of the beam (hereinafter, referred to as an analysis result display portion) is set as the event detection position. The energy distribution display portion C1 shows the energy distribution of the beam passing near the selected electromagnet. The analysis result display portion D1 shows position coordinates of the center of gravity and a size of the beam as an analysis result of the beam shown in the energy distribution display portion C1. As the size of the beam, for example, the size in the horizontal direction and the size in the vertical direction are shown. The display in the analysis result display portion D1 is updated every time the beam in the energy distribution display portion C1 is updated.

In addition, a specific icon E1 in the beam observation application is set as a landmark. The icon E1 used as the landmark has a specific shape different from other portions in the display window B1. The icon E1 is, for example, a camera icon. As illustrated in FIGS. 4 and 5, even if the position of the display window B1 of the beam observation application in the display screen A changes, the relative position of the icon E1 with respect to the energy distribution display portion C1 and the analysis result display portion D1 does not change.

### (Step S101)

Upon starting of the data collection processing, the landmark detection unit 111 continuously acquires image data of the input image A1 displayed on the display of the work terminal 200 from the work terminal 200. The acquired image data is used as an input image.

### (Step S102)

The landmark detection unit 111 detects the icon E1 set as the landmark by image analysis on the input image.

### (Step S103)

Next, the position setting unit 112 reads relative positions of the energy distribution display portion C1 and the analysis result display portion D1 with respect to the icon E1, and determines positions of the energy distribution display portion C1 and the analysis result display portion D1 based on a position of the icon E1 detected in step S102 and the relative positions that have been read.

Then, the position setting unit 112 sets the energy distribution display portion C1 as the log collection range, and sets the analysis result display portion D1 as the event detection position.

### (Step S104)

Next, the event detection unit 113 continuously executes image analysis on the analysis result display portion D1 set as the event detection position, and processing of determining presence or absence of an event based on a result of the analysis. In a case where the coordinates of the center of gravity and the size of a beam displayed in the analysis result display portion D1 change due to a change in the parameter related to the electromagnet, the event detection unit 113 detects the change in the beam as an event.

### (Step S105)

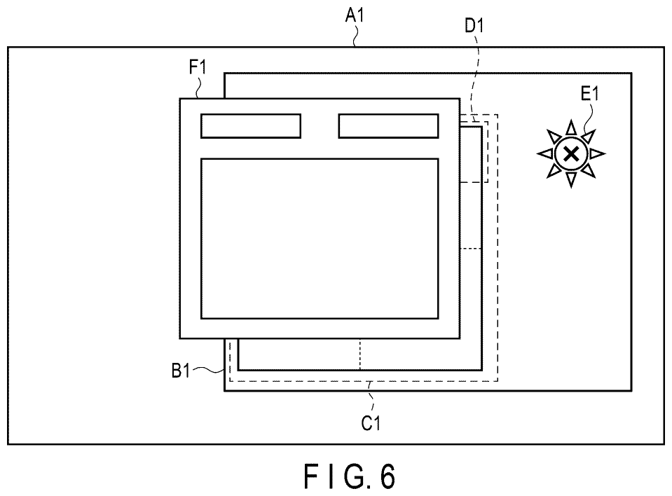

If an event is detected (Yes in step S104), the exception determination unit 114 determines whether or not an image in the energy distribution display portion C1 set as the log collection range in the input image for which the event has been detected is an exception image. For example, as illustrated in FIG. 6, if a display window F of another application is displayed over the energy distribution display portion C1, the exception determination unit 114 determines that an image of the energy distribution display portion C1 is an exception image that is not appropriate as collected data.

### (Step S106)

If an event is detected (Yes in step S104) and the image of the energy distribution display portion C1 in the input image in which the event is detected is not the exception image (No in step S105), the data collection unit 115 stores the image of the energy distribution display portion C1 in the input image in which the event is detected as data of the log collection range.

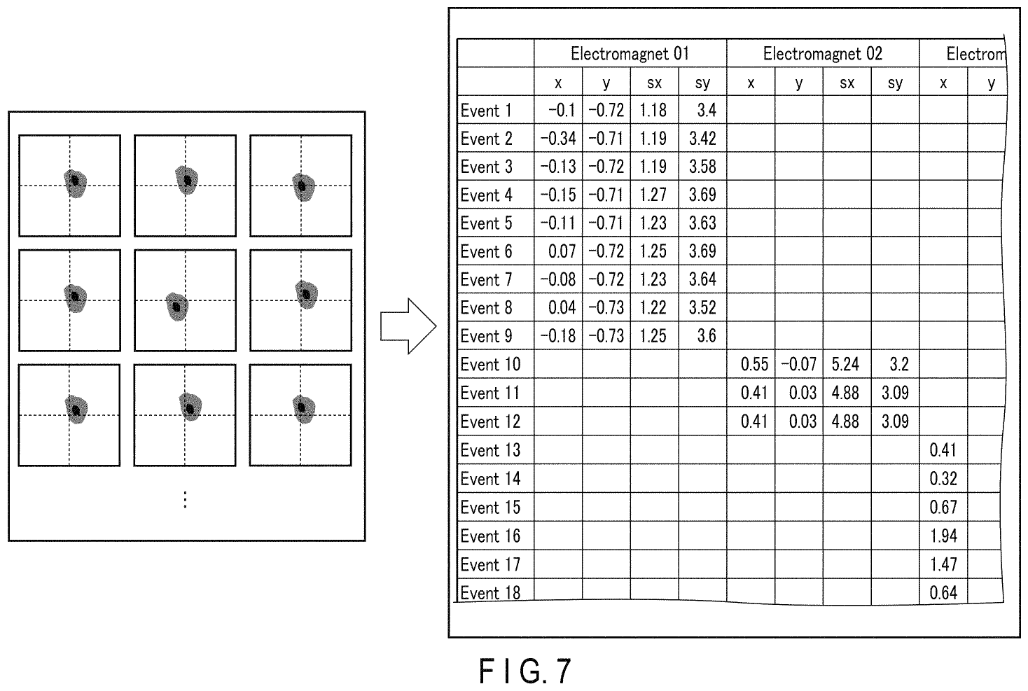

In addition, the data collection unit 115 executes image analysis on the stored image of the energy distribution display portion C1 and calculates the center of gravity and the size of the beam. Then, the data collection unit 115 stores the analysis result for each input image. FIG. 7 is a diagram illustrating a state in which the analysis result of the image of the energy distribution display portion C1 is stored. In FIG. 7, an X-axis coordinate “x” of the center of gravity of the beam, a Y-axis coordinate “y” of the center of gravity of the beam, a beam size “sx” on the X-axis, and a beam size “sy” on the Y-axis are stored for each event that has not been determined as an exception image. In FIG. 7, the analysis result (x, y, sx, sy) is stored for each electromagnet to be adjusted.

### (Step S107)

The processing circuitry 11 repeatedly executes the processing of steps S101 to S106 until the work using the beam observation application in the work terminal 200 is completed (No in step S107), and continuously collects the image of the energy distribution display portion C1 at the time of occurrence of the event as work data. Then, upon completion of the work using the beam observation application (Yes in step S107), the processing circuitry 11 ends a series of data collection processing.

Hereinafter, effects of the data collection apparatus 100 according to the present embodiment will be described.

In a case where the work terminal 200 that performs beam adjustment is highly versatile terminal such as a PC, a place to collect a work log in a screen or a place of interest for detecting a transition of the screen may change depending on a manner of operation by the user. In addition, a display window to be collected may be hidden by a display window of another application. In these cases, data collection may be unsuccessful. Further, while it is preferable to use a numerical value or a beam shape that changes according to a change in a situation as the event detection position for determining a timing of the data collection, but since the numerical value or the beam shape always changes, it is difficult to directly detect the event detection position by image analysis.

The data collection apparatus 100 according to the present embodiment is connected to the work terminal 200 including a display. The data collection apparatus 100 includes the landmark detection unit 111, the position setting unit 112, the event detection unit 113, the exception determination unit 114, and the data collection unit 115. The landmark detection unit 111 detects a landmark from the input image that has been input. The input image is a display image displayed on the display of the work terminal 200. The input image includes a display window that displays the log collection range, the event detection position, and the landmark. The display window is, for example, a display window of a parameter input application or a display window of a beam observation application of the heavy particle therapy apparatus. The log collection range includes, for example, a display area of a work log such as an adjustment target or a parameter. The event is, for example, a change in the log collection range. The relative position of the landmark with respect to the log collection range and the event detection position does not change according to the position of the display window in the input image. In other words, as the landmark, it is possible to use a portion in which the relative position with respect to the log collection range and the event detection position does not change even if the position of the display window of the application changes. For example, a portion having a specific color or shape in the input image is used as the landmark.

The position setting unit 112 sets the log collection range and the event detection position based on the relative positions with respect to the landmark. The event detection unit 113 detects an event at the event detection position. The data collection unit 115 collects image data in the log collection range in a case where the event is detected. At this time, the data collection unit 115 may execute image analysis on the log collection range and collect an analysis result.

According to the above configuration, the data collection apparatus 100 of the present embodiment is able to detect the landmark from the input image output from the work terminal 200, and to determine the log collection range and the event detection position using the relative position with respect to the landmark, and therefore, even if the layout and position of the application are changed by the user's operation, the log collection range and the event detection position can be accurately determined, and the work log can be efficiently collected. In addition, by using an object having a unique color or shape as the landmark, the landmark can be easily detected from the input image.

In addition, the data collection apparatus 100 according to the present embodiment further includes the exception determination unit 114 that executes exception value determination for the image data in the log collection range, and the data collection unit 115 only collects image data that is not determined as the exception value. According to this configuration, the exception value determination is performed on the image within the log collection range, and the work log can be collected by removing an image in a case where the log collection range is hidden by another display or in a case where the log collection range is erroneously set. Therefore, the collection accuracy of the work log is improved. In addition, since unnecessary work logs can be excluded, capacity reduction and cost reduction of stored data can be realized.

Note that the positional relationship between the log collection range and the event detection position with respect to the landmark may change according to an enlargement factor of the display window. In this case, the enlargement factor of the display window may be determined from the size of the detected landmark, and the relative position may be corrected according to the enlargement factor.

Alternatively, only the log collection range may be set using the landmark. In this case, as compared with a case where the log collection range is set without using the landmark, it is possible to suppress setting of an area in which unintended images are mixed as the log collection range and to improve the collection accuracy of the work log. Therefore, capacity reduction and cost reduction of stored data can be realized. Alternatively, only the event detection position may be set using a landmark. In this case, as compared with a case where the event detection position is set without using the landmark, it is possible to effectively detect a change in the log collection range, and to improve the collection accuracy of the work log. Therefore, capacity reduction and cost reduction of stored data can be realized.

Specifically, according to the data collection apparatus 100 of the present embodiment, it is possible to improve the collection accuracy of the work log by setting a detection position (the log collection range or the event detection position) for determining a collection range of the work log (the collection position or the collection timing) based on the relative position with respect to the landmark.

### (First Modification)

In the above embodiment, the case of collecting the work log of the beam observation application has been mainly described. In the present modification, a case of collecting a work log of the parameter input application will be described. In the present modification, the work terminal 200 is a terminal that changes a parameter of each electromagnet of the heavy particle therapy apparatus using the parameter input application, and the parameter input by the user is collected using the data collection apparatus 100.

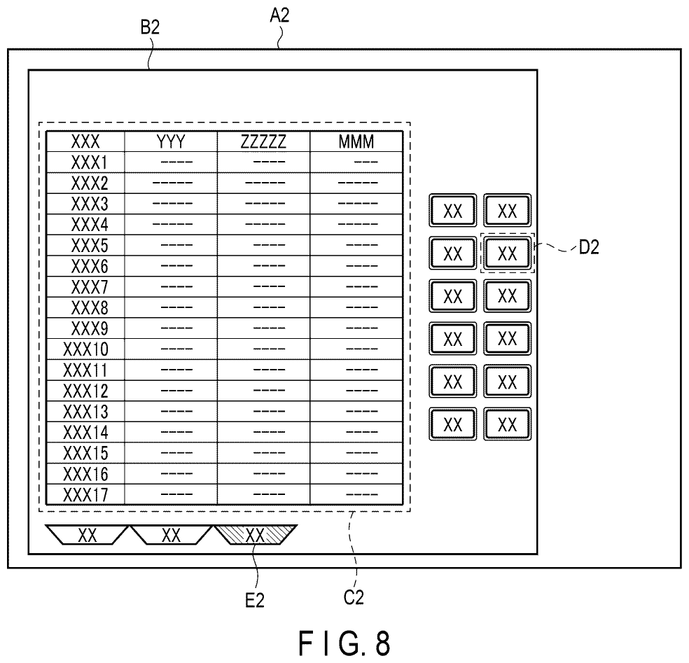

FIG. 8 is a diagram illustrating an example of an input image A2 output to the display of the work terminal 200 in the present modification. In the example of FIG. 8, a display window B2 of the parameter input application is displayed in the input image A2.

Here, a parameter input portion C2 for inputting a parameter is set as the log collection range, and an operation button D2 for parameter change is set as the event detection position. In the parameter input portion C2, a setting value of a parameter related to the selected electromagnet. Each parameter displayed in the parameter input portion C2 can be changed by the user's input. The operation button D2 is a setting button for setting the parameter input in the parameter input portion C2. Upon selection of the operation button D2, the current parameter is updated to the parameter displayed in the parameter input portion C2, and the color or shape of the operation button D2 changes.

In addition, a specific tab E2 in the parameter input application is set as a landmark. The tab E2 used as the landmark has a specific color different from other portions in the display window B2. For example, only the tab E2 is displayed in black in the display window B2. As illustrated in FIG. 8, even if the position of the display window B2 of the parameter input application on the display screen A changes, the relative position of the tab E2 with respect to the parameter input portion C2 and the operation button D2 does not change.

In the present modification, in the process of step S104, the event detection unit 113 continuously executes image analysis on the operation button D2 set as the event detection position, and processing of determining presence or absence of an event based on a result of the analysis. In a case where the operation button D2 changes to a state indicating that the operation has been performed, the event detection unit 113 determines that the parameter related to the electromagnet has been updated, and detects the update of the parameter as an event.

Further, in the processing of step S106, the data collection unit 115 stores the image of the parameter input portion C2 in the input image in which the event is detected as data of the log collection range. At this time, the data collection unit 115 executes image analysis on the stored image of the parameter input portion C2 to detect the parameter, and stores an analysis result for each input image. As a result, every time the operation button D2 which is a setting button is selected and the parameter of the electromagnet is updated, the updated parameter can be collected.

### (Second Modification)

In the present modification, a case where work logs of both the beam confirmation application and the parameter input application are collected will be described. In the present modification, the worker uses the work terminal 200 to change the parameter of each electromagnet of the heavy particle therapy apparatus using the parameter input application, and to observe the heavy particle beam passing near each electromagnet of the heavy particle therapy apparatus using the beam observation application. The data collection apparatus 100 collects a parameter set using the parameter input application and an energy distribution of the heavy particle beam in a case in which the parameters are updated.

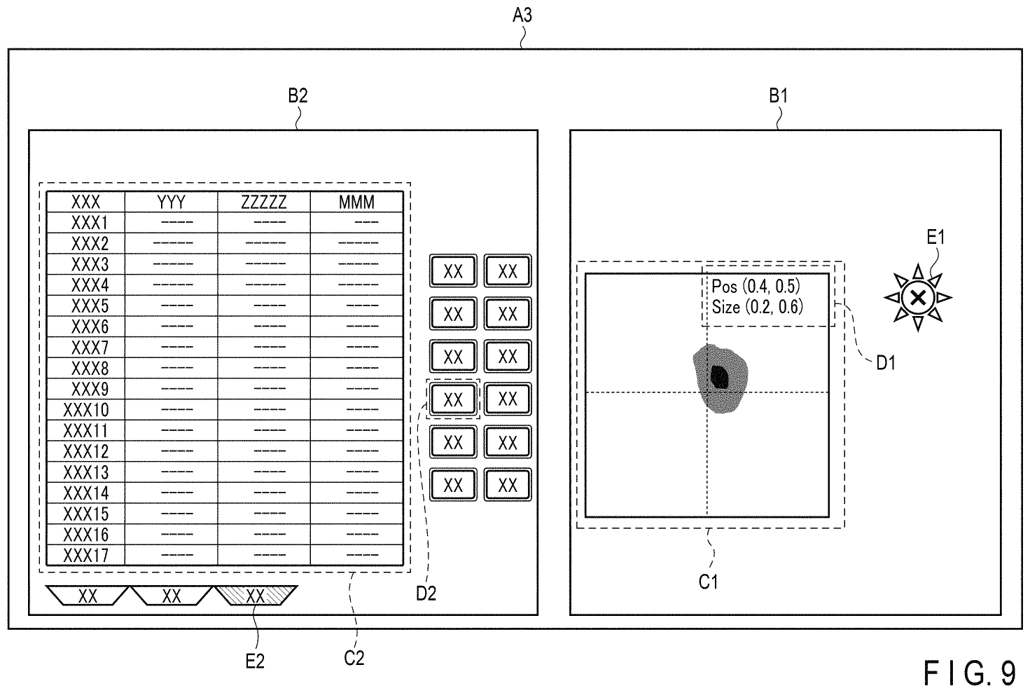

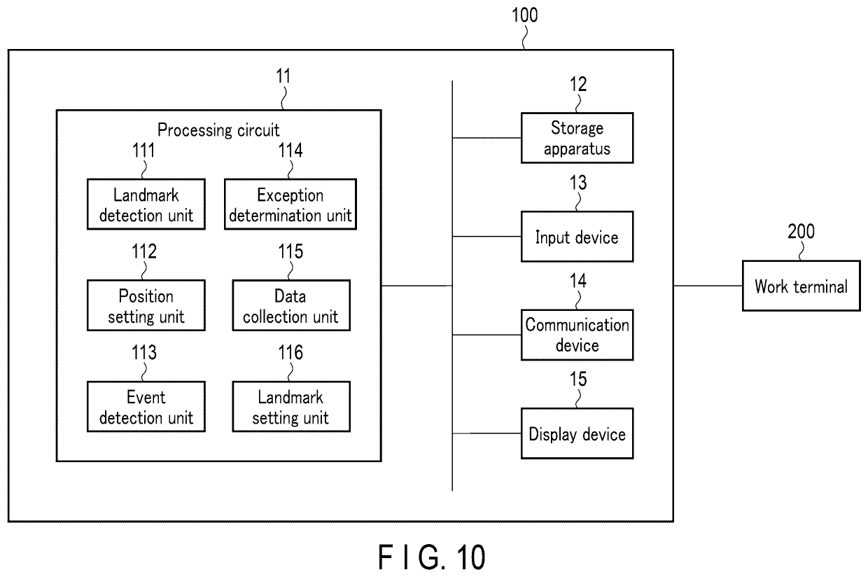

FIG. 9 is a diagram illustrating an example of an input image A3 output to the display of the work terminal 200 in the present modification. In the example of FIG. 9, a display window B1 of the observation application and the display window B2 of the parameter input application are displayed in the input image A3. Since the display window B1 of the observation application is similar to that described with reference to FIGS. 4 and 5, and the display window B2 of the parameter input application is similar to that described with reference to FIG. 8, a detailed description thereof will be omitted.

In the present modification, in step S102, the landmark is detected from the input image A3. At this time, the landmark detection unit 111 determines that both the parameter input application and the beam confirmation application are displayed based on the detection of the icon E1 and the tab E2 as the landmarks. Thereafter, in step S103, the energy distribution display portion C1 and the parameter input portion C2 are set as the log collection ranges, and the analysis result display portion D1 and the operation button D2 are set as the event detection positions.

Then, in step S104, in a case where the operation button D2 changes to a state indicating that the operation has been performed, it is determined that an event has occurred. In this case, in step S106, the data collection unit 115 stores the image of the parameter input portion C2 and the set value of the parameter obtained by image analysis.

In addition, in a case where the operation button D2 is pressed and the parameter is changed, a voltage or the like of the electromagnet gradually changes, a change gradually appears in the energy distribution display portion C1 in the beam confirmation application, and the coordinates of the center of gravity and the size of the beam displayed in the analysis result display portion D1 change. In step S104, in a case where the coordinates of the center of gravity and the size of the beam displayed in the analysis result display portion D1 are changed, it is determined that an event has occurred, and in step S106, the image of the energy distribution display portion C1 and the position and the size of the heavy particle beam obtained by the image analysis are stored. At this time, the data collection unit 115 stores the position and the size of the heavy particle beam acquired from the energy distribution display portion C1 in association with the set value of the parameter acquired from the parameter input portion C2.

If it is determined in step S105 that the image of the energy distribution display portion C1 or the image of the parameter input portion C2 is the exception image, the image is not stored.

According to the present modification, every time the parameter of the electromagnet is updated, the position and the size of the heavy particle beam can be stored in association with the parameter.

Note that data collection may be performed simultaneously in both the beam confirmation application and the parameter input application at the timing at which an event is detected in one of the beam confirmation application and the parameter input application. In this case, the detection of the event may be performed by only one of the beam confirmation application and the parameter input application.

### Second Embodiment

A second embodiment will be described. The present embodiment is obtained by modifying the configuration of the first embodiment as follows. Description of configurations, operations, and effects similar to those of the first embodiment will be omitted. In the first embodiment, a preset landmark is used. In the present embodiment, the landmark is automatically set using an input image.

FIG. 10 is a diagram illustrating a configuration example of a data collection apparatus 100 according to the present embodiment. As illustrated in FIG. 10, a processing circuitry 11 further includes a landmark setting unit 116. The processing circuitry 11 further realizes a landmark setting function by executing a program.

The landmark setting unit 116 automatically sets the landmark based on the image for setting the landmark (hereinafter, referred to as a setting image). The setting image is an image of the same type as the input image, and is input by the user, for example. The landmark setting unit 116 detects a unique pattern in the setting image that has been input, and automatically sets the detected unique pattern as the landmark. Note that an image group included in the moving image data may be used as the setting image.

For example, the landmark can be set by using the fact that a correlation between the unique pattern and other regions in the input image is low. In this case, the landmark setting unit 116 calculates, for each pixel of the setting image, an average of inverses of the correlation with other positions as a degree of uniqueness, detects a region where pixels having a large degree of uniqueness are gathered as a unique pattern, and sets the detected region as the landmark.

Alternatively, an area that is detected as the unique pattern and in which a relative position with respect to the specified log collection range and the event detection position does not change may be set as the landmark. In this case, the user inputs a plurality of setting images in which the position of the display window of the application on the image has changed, and designates the log collection range and the event detection position in each setting image. The landmark setting unit 116 detects, in each setting image, a region in which the positional relationship with the log collection range and the event detection position does not change and in which the degree of uniqueness is large, and sets the detected region as the landmark.

### (Data Collection Processing)

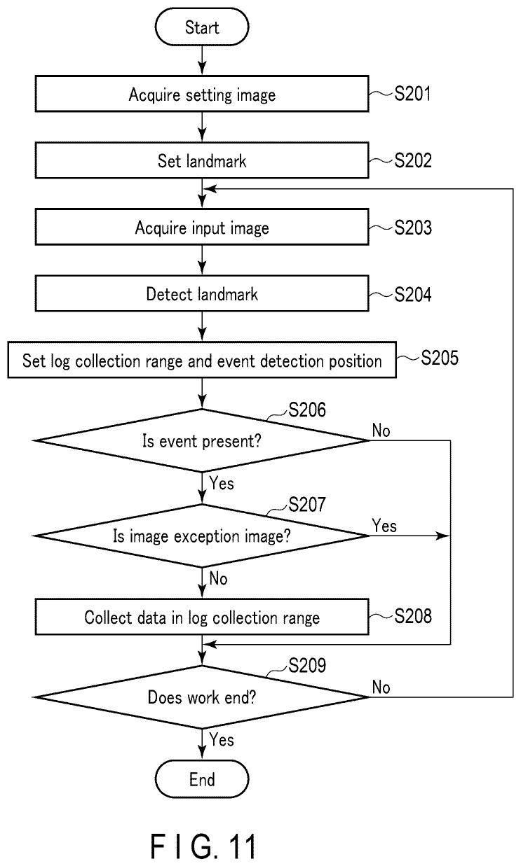

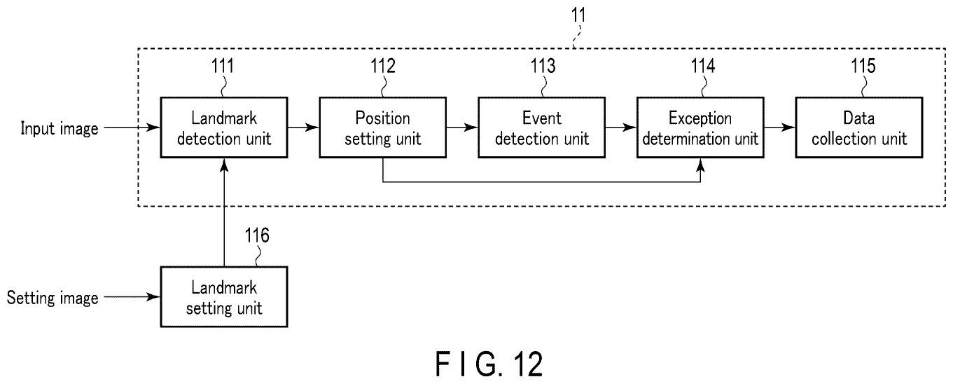

Next, an operation of the data collection processing executed by the data collection apparatus 100 according to the present embodiment will be described. FIG. 11 is a flowchart showing an example of a procedure of the data collection processing. FIG. 12 is a diagram illustrating an example of a flow of data in the data collection processing.

In the present modification, first, the user inputs the setting image to the data collection apparatus 100. The landmark setting unit 116 acquires the setting image that has been input (step S201), performs image analysis on the setting image, detects a unique pattern in the setting image, and sets the detected unique pattern as the landmark (step S202).

Then, the work log of a work terminal 200 can be collected using the automatically set landmark by the processing of steps S203 to S209. Since the processing in steps S203 to S209 is similar to the processing in steps S101 to S107 in FIG. 2, detailed description thereof is omitted.

According to the data collection apparatus 100 of the present embodiment, it is possible to collect work logs using automatically set landmarks without setting icons and tabs suitable as landmarks in advance, and it is possible to reduce a burden on the user.

Thus, according to any one of the above-described embodiments, it is possible to provide a data collection apparatus capable of improving the collection accuracy of the work log, a method, and a program.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A data collection apparatus comprising a processing circuitry that is configured to:
detect a landmark from an input image that has been input;
set, based on a relative position with respect to the landmark, either a log collection range or an event detection position;
detect an event at the event detection position; and
collect data of the log collection range in a case where the event is detected.

2. The data collection apparatus according to claim 1, wherein
the processing circuitry is configured to:
execute exception value determination on an image of the log collection range; and
collect only an image that is not determined to have an exception value.

3. The data collection apparatus according to claim 1, wherein
the input image includes a display window showing the log collection range, the event detection position, and the landmark, and
a relative position of the landmark with respect to the log collection range and the event detection position does not change according to a position of the display window in the input image.

4. The data collection apparatus according to claim 3, wherein
the landmark is a unique pattern having a specific color or shape in the input image.

5. The data collection apparatus according to claim 1, wherein
the log collection range includes a display area of a work log, and
the event is a change in the log collection range.

6. The data collection apparatus according to claim 1, wherein
the processing circuitry is configured to execute image analysis on the log collection range, and collect an analysis result.

7. The data collection apparatus according to claim 1, wherein
the data collection apparatus is connected to a work terminal having a display, and
the input image is a display image displayed on the display of the work terminal.

8. The data collection apparatus according to claim 1, wherein
the processing circuitry is configured to set the landmark based on the input image.

9. A data collection apparatus comprising a processing circuitry that is configured to:
detect a landmark from an input image acquired from a heavy particle therapy apparatus that emits a heavy particle beam using an electromagnet;
based on a relative position with respect to the landmark, set an area in which an energy distribution of the heavy particle beam near the electromagnet is displayed as a log collection range, and set an area in which an analysis result of the heavy particle beam is displayed as an event detection position;
detect a change in the analysis result at the event detection position as an event; and
analyze, in a case where the event is detected, the energy distribution of the heavy particle beam in the log collection range, and collect the analysis result as data of the log collection range.

10. A data collection apparatus comprising a processing circuitry that is configured to:
detect a landmark from an input image acquired from a heavy particle therapy apparatus that emits a heavy particle beam using an electromagnet;
based on a relative position with respect to the landmark, set an area in which a parameter related to the electromagnet is displayed as a log collection range, and set an operation unit operated in a case where the parameter is changed as an event detection position;
detect a change in the operation unit at the event detection position as an event; and
collect, in a case where the event is detected, the parameter displayed in the log collection range as data of the log collection range.

11. The data collection apparatus according to claim 9, wherein
the processing circuitry is configured to:
execute exception value determination on an image of the log collection range; and
collect only an image that is not determined to have an exception value.

12. The data collection apparatus according to claim 9, wherein
the input image includes a display window showing the log collection range, the event detection position, and the landmark, and
a relative position of the landmark with respect to the log collection range and the event detection position does not change according to a position of the display window in the input image.

13. The data collection apparatus according to claim 12, wherein
the landmark is a unique pattern having a specific color or shape in the input image.

14. A method comprising:
detecting a landmark from an input image that has been input;
setting, based on a relative position with respect to the landmark, either a log collection range or an event detection position;
detecting an event at the event detection position; and
collecting image data of the log collection range in a case where the event is detected.

15. A non-transitory computer-readable storage medium storing a program for causing a computer to execute:
a function of detecting a landmark from an input image that has been input;
a function of setting, based on a relative position with respect to the landmark, either a log collection range or an event detection position;
a function of detecting an event at the event detection position; and
a function of collecting image data of the log collection range in a case where the event is detected.
